(19) United States
(12) Plant Patent Application Publication
(10) Pub. No.: US 2004/0181838 P1
(43) Pub. Date: Sep. 16, 2004

(54) Kalanchoe thyrsiflora plant named 'Variegated Desert Rose'

(76) Inventor: Mariska van Ruyven, Naaldwijk (NL)

Correspondence Address: Paul S. Rooy, 2620 S. Peninsula Dr., Daytona Beach, FL, 32118, US

(21) Appl. No.: 10/733,743

(22) Filed: Dec. 11, 2003

(30) Foreign Application Priority Data
Mar. 14, 2003 (NL) 2003/0465

(51) Int. Cl. A01H 5/00

(52) U.S. Cl. PLT/339

(57) ABSTRACT

Kalanchoe thyrsiflora plant named ‘Variegated Desert Rose’. ‘Variegated Desert Rose’ is characterized by variegated grayish-green leaves, with yellow parts comprising 30%-80% of the leaf surface. The leaves may have a red border if ‘Variegated Desert Rose’ is grown in full sun or a dry environment.

### LATIN NAME OF THE GENUS AND SPECIES OF THE PLANT CLAIMED

The plant genus is Kalanchoe. The plant species is thyrsiflora.

### FIELD OF THE INVENTION

The present invention comprises a new and distinct cultivar of Kalanchoe thyrsiflora which is named ‘Variegated Desert Rose’. Its genus is Kalanchoe. Its market class is that of ornamental potted plants.

### DESCRIPTION OF PRIOR ART

‘Variegated Desert Rose’ is the product of a planned breeding program carried out in 2001 and 2002 in Naaldwijk, the Netherlands. It is a seedling of a selfing line of Kalanchoe thyrsiflora Desert Rose. That is, the original ‘Variegated Desert Rose’ was the result of a crossing where the mother and the father were flowers from the same plant. The crossing was made by Mariska van Ruyven in 2001.

Uniformity:

Kalanchoe thyrsiflora ‘Variegated Desert Rose’ was first asexually reproduced in 2002 by cuttings. The plant is stable as evidenced by propagation over three successive generations, using rooting cuttings, over a period of more than 18 months. The first generation was comprised of approximately 40 plants, the second generation was comprised of approximately 150 plants, and the third generation is in progress. Propagation was carried out in Naaldwijk, the Netherlands.

No off-types have been observed throughout the trial period. The variety is considered to be uniform and stable in all characteristics.

### SUMMARY OF THE INVENTION

The major characteristics of ‘Variegated Desert Rose’ include thick variegated leaves having a lot of yellow coloration. The plant itself is a succulent.

The following traits which have been repeatedly observed are determined to be basic characteristics of ‘Variegated Desert Rose’, which in combination distinguish ‘Variegated Desert Rose’ from other Kalanchoe thyrsiflora of the same general type.

1. Plant growth habit is compact: height 30 cm at maturity.

2. Only a few branches are produced.

3. The internodes are about 1 cm, depending on the light. More light will produce shorter internodes.

4. Leaves are thick and every leaf contains a bud.

5. Leaves are variegated yellow 4C on green 147B (R.H.S. Colour Chart), with the yellow part comprising 30%-80% of the surface of the leaf.

6. When grown in full sunshine or a dry medium, the leaves become red-edged. The size of the red edge depends on the light or draught circumstances: the more light or dryness, the larger the red edge.

7.The seeds are very small (1 mm max).

### BRIEF DESCRIPTION OF THE DRAWINGS

The invention, together with the other objects, features, aspects and advantages thereof will be more clearly understood from the following in conjunction with the accompanying drawings.

Four sheets of drawings are provided. Sheet one contains FIG. 1. Sheet two contains FIG. 2. Sheet three contains FIG. 3. Sheet 4 contains FIG. 4.

FIG. 1 is a color photograph of a front view of ‘Variegated Desert Rose’ plant.

FIG. 2 is a color photograph of a top view of ‘Variegated Desert Rose’ plant.

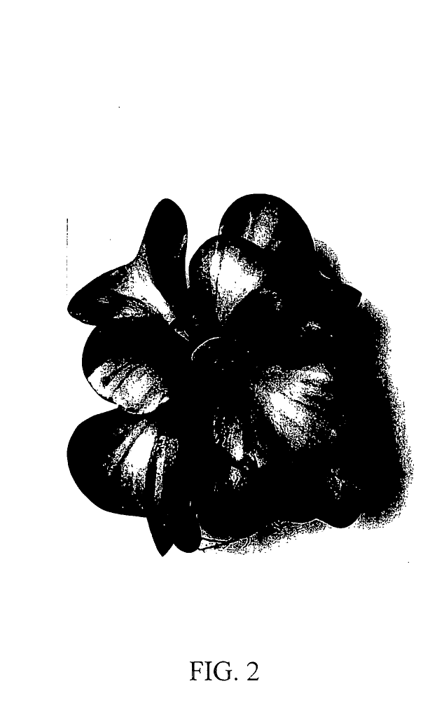

FIG. 3 is a color photograph of a group of seedlings from the selfing of ‘Desert Rose’, in which ‘Variegated Desert Rose’ was found.

FIG. 4 is a color photograph close-up view of three ‘Variegated Desert Ros” leaves.

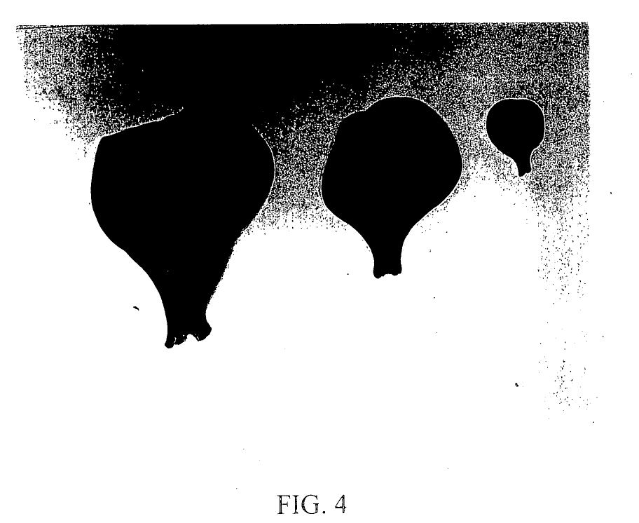

### BOTANICAL DESCRIPTION OF THE PLANT

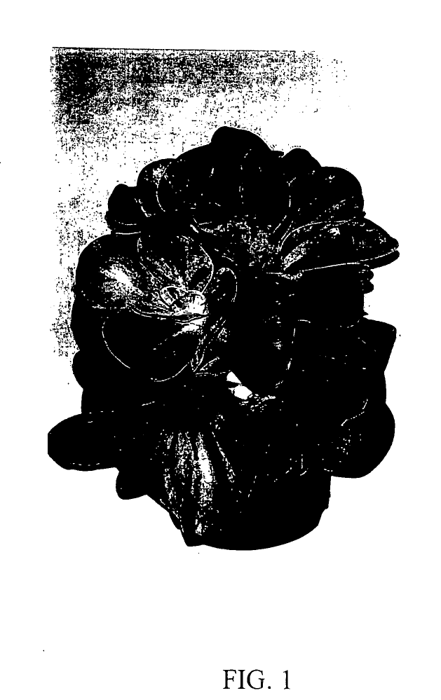

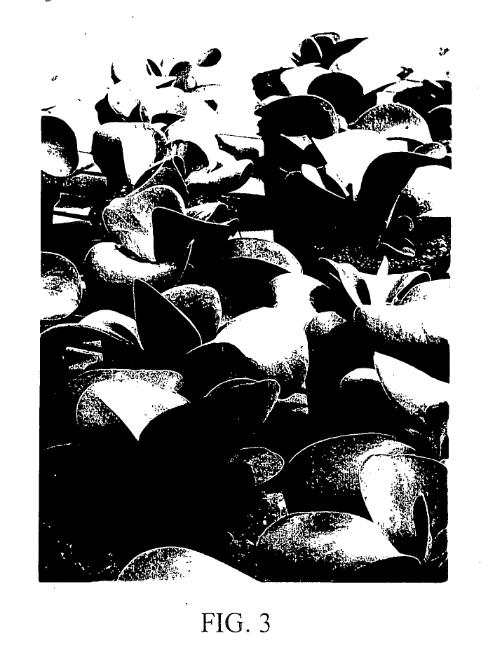

FIGS. 1 depicts an eighteen-month old Kalanchoe thyrsiflora ‘Variegated Desert Rose’ in a pot grown from a rooted cutting. This ‘Variegated Desert Rose’ was about 30 cm high measured from soil surface to the tip, and about 20 cm wide. FIG. 3 depicts multiple 2 month-old seedlings of a selfing Kalanchoe thyrsiflora ‘Desert Rose’.

The following Botanical Description describes Kalanchoe thyrsiflora ‘Variegated Desert Rose’ plants grown in Naaldwijk, the Netherlands. The plant has not been observed under all possible environmental conditions. The phenotype may vary somewhat with variations in environment such as temperature, light intensity and/or fertilizer rate, without, however, any variance in genotype.

Stem: Growth Pattern: The mature stem is approximately 1-2 cm in diameter about 5 cm above the soil level. Inter-node distance is approximately 1 cm at soil level in mature stems; this distance decreases with increased light.

Color.—The immature stem emerges 190D R.H.S. and darkens to 189D R.H.S. at maturity.

Leaves: Kalanchoe thyrsiflora ‘Variegated Desert Rose’ has heart-shaped leaves. The leaves are grayish green R.H.S. 147B with yellow R.H.S. 4C variegations. At maturity, each leaf contains a bud. In addition, the leaves are covered with a gray R.H.S. 188D powder.

The leaf is substantially symmetrical.—The primary veins are almost invisible.

Growth cycle: The Kalanchoe thyrsiflora ‘Variegated Desert Rose’ plant is grown from rooted cuttings. Within two weeks the cuttings are rooted. Every four weeks the plant forms two new leaves on opposite sides of the stem. Each leaf contains a bud. The Kalanchoe thyrsiflora ‘Variegated Desert Rose’ plants are large enough to sell as mature plants 18-20 weeks from rooting. As in all Kalanchoe thyrsiflora plants, ‘Variegated Desert Rose’ is susceptible to powdery mildew.

All color codes refer to “The Royal Horticultural Society London, 1995 Ed.”. Colors and leaf size may vary somewhat depending on horticultural practices such as light levels and fertilizer rates, among other things, without however, any variance in genotype.

I claim:

1. A new and distinct cultivar of Kalanchoe thyrsiflora plant named ‘Variegated Desert Rose’, as herein described and illustrated.
